(19) United States
(12) Patent Application Publication
(10) Pub. No.: US 2019/0060109 A1
(43) Pub. Date: Feb. 28, 2019

(54) Method of Preventing Traumatic Brain Injury (TBI)

(71) Applicant: Gregory Todd Johnson, Ravena, NY (US)

(72) Inventor: Gregory Todd Johnson, Ravena, NY (US)

(21) Appl. No.: 15/693,239

(22) Filed: Aug. 31, 2017

(51) Int. Cl. A61F 7/02

(52) U.S. Cl. CPC A61F 7/02; A61F 2007/0002

(57) ABSTRACT

This invention is a method for preventing, or reducing the severity of, traumatic brain injury (TBI) by increasing the viscosity of cerebrospinal fluid (CSF) to enhance an existing protective mechanism of the body. The invention increases the viscosity of the cerebrospinal fluid (CSF) that surrounds the brain such that the CSF fluid provides a greater resistance to the movement of the brain to slow its movement relative to the skull during an impact event, thereby reducing the magnitude of acceleration of the brain relative to the skull. Consequently, this method can reduce the severity of impacts on the brain, thereby reducing the potential damage associated with said impacts.

### CROSS-REFERENCE TO RELATED APPLICATIONS

This patent application does not claim the benefit of any prior applications.

### STATEMENT REGARDING FEDERALLY SPONSORED RESEARCH

The development of this invention did not rely on federally sponsored research.

### REFERENCE TO A SEQUENCE LISTING

Not Applicable

### BACKGROUND OF THE INVENTION

One cause of traumatic brain injury (TBI) is the contact of the brain with the inside of the skull, as may occur during impact scenarios such as collisions and falls by sports athletes, high-risk activities such as motorcycling, or explosions in the vicinity of military personnel. Statistics vary by sources, but is it estimated that millions of people suffer some form of TBI event each year (Brain Injury Association of America).

The magnitude of acceleration is one of the primary factors in determining the severity of TBI and the resulting effects (Meaney et. al.). The brain has certain mechanical protective measures in place in order to protect the brain during events that impose acceleration on the brain. Cerebrospinal fluid (CSF) surrounds the brain and provides a fluid cushion to protect the brain during severe movements (Guyton & Hall). The viscous characteristic of fluid gives the fluid an internal friction that resists the motion of objects in that fluid, thereby reducing the velocity of said objects relative to other surrounding objects. Said reduction of velocity results in a reduction in the magnitude of acceleration experienced by the brain when it impacts the skull.

To date, the focus of prior art in the field of TBI has been applied to:
a) The diagnosis, treatment, and recovery of existing TBI conditions. Said prior art includes methods or neuroprotective agents that are intended to mitigate the severity of progression and degeneration after a brain injury event has already occurred. Reference U.S. Pat. Nos. 9,445,991; 9,415,063; 9,365,550; 9,364,432; 8,633,165.
b) Computer modelling of impact events and the effects on the brain for the purpose of injury assessment and prediction of outcomes. Medical literature has identified differences in damage potential due to different mechanical properties of CSF (Baeck et al; Yang et al). However, the research conclusions focused the on need to refine the definition of CSF mechanical properties used in simulation models with the objective of achieving a more accurate model of brain injuries, rather than recognizing that medically significant differences in CSF mechanical properties could be applied for preventive benefits. Medical literature has also acknowledged the potential for CSF viscosity to be a factor affecting individual differences in TBI susceptibility, but this is presented as a speculative possibility that was specifically based on the hydration condition of an athlete, and makes no attempt to explain a method of reducing susceptibility to TBI (Eckner et al).
c) Technological advancements external to the body, such as advanced helmets, to reduce impact forces for preventive purposes. Reference U.S. Pat. Nos. 9,380,823; 9,370,214; 9,314,061; 7,076,811; and U.S. Pat. Application No. 20070186329.
d) The use of apparatus external to the head to monitor forces experienced by the head or to monitor the condition of external protective features. Reference U.S. Pat. Application No. 20160320278.
e) The use of apparatus external to the head to monitor or regulate temperatures with the objective of avoiding heat exhaustion, heat stroke, or heat related death, said objectives pursued by U.S. Pat. Nos. 9,501,918; 9,451,795; 8,544,115; 7,010,813; 6,932,150; 6,904,618. All such technologies are unrelated to the specific beneficial impact of temperature on CSF viscosity for TBI prevention purposes.
f) Temperature-regulating helmet advancements to promote the use of a helmet in those recreational or professional activities in which the wearing of a helmet is desirable for safety reasons or is legally required, U.S. Pat. No. 7,827,620. Such advancements are based upon increasing the comfort level of the wearer so that said wearer will be more likely to wear the helmet during risky activities, rather than any specific preventive benefits associated with reducing the temperature of CSF.

The focus of prior art specific to the field of CSF has generally been the analysis of CSF for the purpose of diagnosing existing or developing nervous system conditions, U.S. Pat. No. 7,033,573. In the course of research for this application, no prior art could be found in the patent literature which used intentional adjustments of the viscosity of CSF prior to an impact event for the purpose of TBI prevention.

### BRIEF SUMMARY OF THE INVENTION

The proposed invention is a method to prevent, or reduce the severity of, traumatic brain injury (TBI) by increasing the viscosity of cerebrospinal fluid (CSF). CSF surrounds the brain and provides a fluid layer to protect the brain during impact events which introduce a motion of the brain relative to the skull. Currently accepted TBI assessment methods identify the magnitude of acceleration of the brain as one primary determinant of the severity of TBI injury. The proposed claims involve modification of the viscosity characteristic of CSF such that CSF provides a greater resistance to motion of the brain relative to the skull during an event, thereby reducing the velocity of the brain relative to the skull and the resulting magnitude of acceleration of the brain when the brain impacts the skull. Claims include the implementation of the proposed method by controlling the temperature of the CSF in and around the brain, by increasing the concentration of certain constituents of CSF, or by manipulating the behavior of certain specific bodily structures which affect CSF creation and CSF circulation.

### BRIEF DESCRIPTION OF DRAWINGS

FIG. 1 depicts certain human features related to CSF and its location and general circulation path in the central nervous system. This includes the generation of CSF in the areas of the choroid plexus, arrows to depict CSF flow, and its path out of the central nervous system via the arachnoid villi.

FIGS. 2 and 3 depict of the mechanical concepts associated with the invention. Said figures are presented only to depict the concept of the invention, and are not shown to scale.

FIG. 2 depicts the movement of the brain relative to the skull and the motion of CSF during an impact event that induces rotational motion of the brain relative to the skull. FIG. 2 depicts decreasing velocities of CSF layers between the brain and the skull. This velocity profile results from the fluid friction between layers of CSF which resists the movement of CSF, and consequently, resists the movement of the brain. A higher viscosity of CSF will result in lower velocities, and consequently a lower magnitude of acceleration, of the brain relative to the skull. This figure is independent of the particular type or shape of physical object, force, or wave that initiates the movement of the brain relative to the skull.

FIG. 3 depicts the movement of the brain relative to the skull and the motion of CSF during an impact event that induces linear motion of the brain relative to the skull. FIG. 3 depicts the motion of the brain toward the skull, and the resulting displacement of CSF out of the space between the brain and the skull. A higher viscosity of CSF will provide a greater resistance to the displacement of CSF and will result in a lower velocity, and consequently a lower acceleration, of the brain relative to the skull. This figure is independent of the particular type or shape of physical object, force, or wave that initiates the movement of the brain relative to the skull.

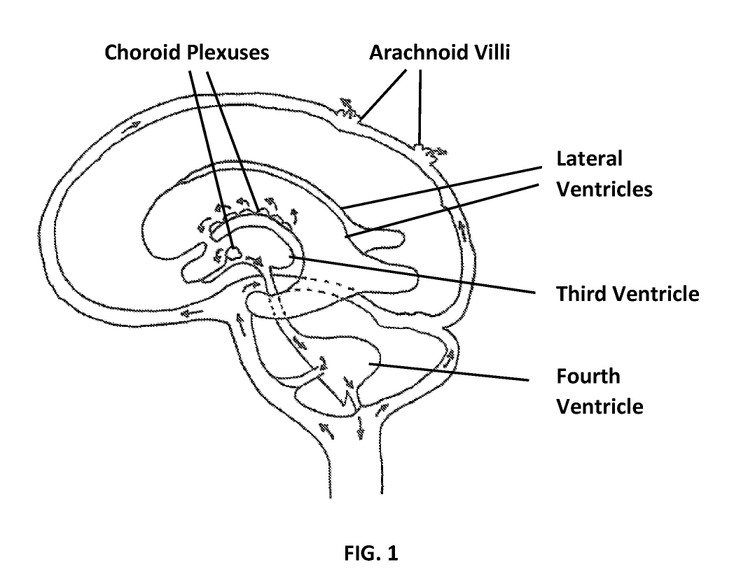

### DETAILED DESCRIPTION OF THE INVENTION

“Impact” is used in the discussion and illustration of situations related to TBI and CSF viscosity. The use of said term is for demonstration purposes in order to convey the concept of the invention, and is intended to encompass any event in which the brain experiences movement relative to the skull. The use of said terms is not intended to bound or imply every possible situation or condition in which a change in CSF viscosity could positively affect outcomes during a potentially harmful event, and is not intended to limit the potential applications of the claims herein. In addition, the demonstration of impact discussed herein is valid regardless of whether the relative movement of the brain and skull results from a coup or a contrecoup injury scenario.

An “increase” in the viscosity of CSF as used herein also refers to the opposition of conditions that would otherwise decrease the viscosity of CSF. For example, an embodiment of this invention is considered to increase the viscosity of CSF if that embodiment utilizes temperature control to maintain the CSF viscosity associated with a normal body temperature level even if current bodily conditions or ambient temperature conditions would have resulted in a lower CSF viscosity. In other words, the avoidance of viscosity decreases is considered to be one form of an “increase” of CSF viscosity.

“Magnitude of acceleration” is used throughout this application to identify the acceleration of the brain relative to the surrounding skull. Said term includes negative values of acceleration, also known as deceleration.

“Traumatic brain injury” and “TBI” are used to identify any injury condition that occurs when an external mechanical force affects brain function. This condition typically occurs during impact scenarios in which the physical structure of the skull imposes a force on brain tissue. It is acknowledged that there are certain non-rigid tissues in the human body between the brain matter and the skull bone, such as the meninges. Contact between the brain and skull, as used herein, refers to the scenarios in which the magnitude of acceleration of the brain relative to the skull is such that said tissues between the brain and skull have compressed to the point where the skull structure imposes a potentially damaging magnitude of force against brain tissue to resist the movement of said brain tissue. It is acknowledged that certain forms of TBI could occur without actual contact between the brain and the skull. The proposed invention encompasses all conditions under which the magnitude of acceleration of the brain relative to the skull is reduced due to increased CSF viscosity, up to and including coup, contrecoup, and other contact between the brain and the skull. The invention is not dependent on any degree of actual contact, and does not depend on any medical differences between the terms TBI, mild TBI, and concussion.

All stated embodiments of the invention identified herein are illustrative of particular embodiments of the invention, and are not to be construed as limiting the invention of the claimed method. Embodiments discussed herein are not necessarily exclusive and shall not be construed as excluding any combinations with other embodiments.

Cerebrospinal fluid (CSF) consists of approximately 150 mL of liquid which surrounds the brain and spinal cord, and among other functions, provides mechanical protection to cushion the brain. Approximately 500 mL of CSF is produced each day by the average adult human, meaning that CSF turnover is over 3 times per day. Most CSF is produced by secretion through the choroid plexus, and additional production comes from secretions by ependymal surfaces of the ventricles and arachnoid membranes. The vast majority of CSF consists of water, but it also includes measureable amounts of salts, sugars, proteins, and minerals. CSF circulates throughout ventricles and subarachnoid spaces around the brain and spinal cord cavity. Most CSF is believed to exit the brain cavity and pass into the circulatory system primarily through the arachnoid villi (Guyton & Hall).

The viscosity of a fluid is a measure of its resistance to deformation. This resistance to deformation manifests itself in the fluid friction between CSF layers in a rotational impact scenario and in the resistance to CSF displacement in a linear impact scenario. The invention increases the viscosity of CSF such that the CSF imposes a greater resistance to movement of the brain relative to the skull during an impact event, thereby reducing the maximum velocity of the brain relative to the skull and therefore reducing the magnitude of acceleration experienced by the brain relative to the skull. Linear and rotational acceleration have been identified as important biomechanical factors in the mechanics of brain injury and in determining the severity of TBI during an impact event.

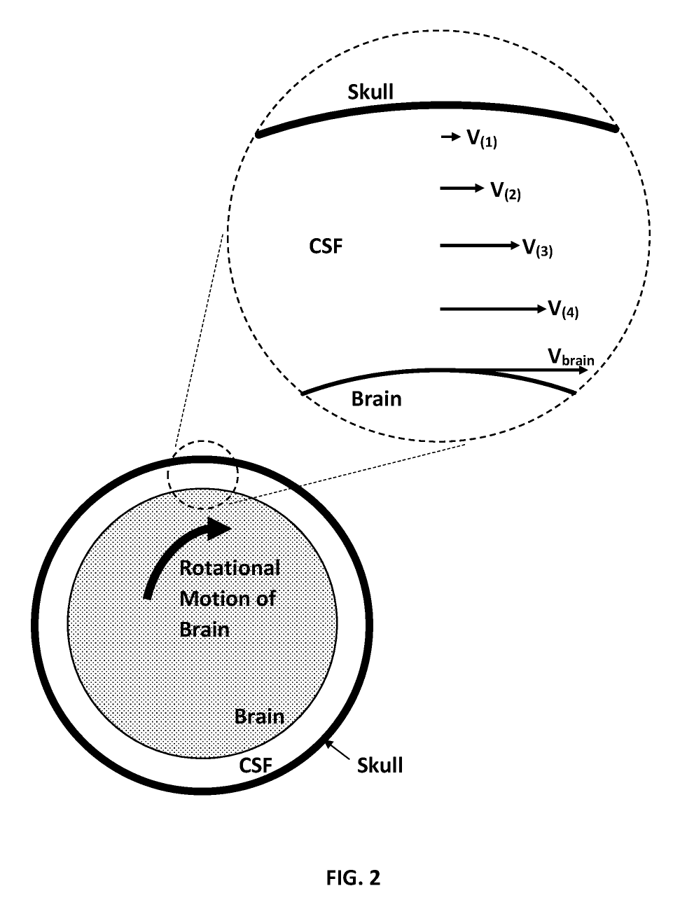

In a rotational example, the motion of the brain relative to the skull is dependent on the viscosity of the fluid. A simplified diagram of rotational brain movement is depicted in FIG. 2. In said simplified case, the motion of the brain is opposed by a friction force imposed on the brain by CSF.

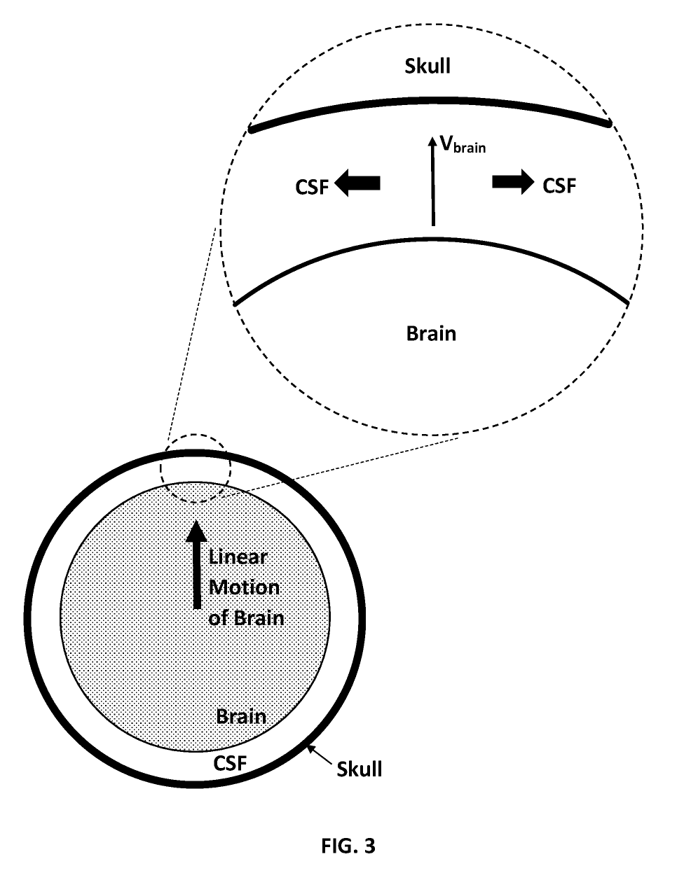

In a linear example, the motion of the brain relative to the skull is dependent on the viscosity of the fluid. A simplified diagram of linear brain movement is depicted in FIG. 3. In said simplified case, the motion of the brain is resisted by the displacement of CSF as the brain moves toward the skull, consequently reducing the velocity of the brain relative to the skull.

In the said rotational and linear cases, a higher CSF viscosity will result in a larger force being exerted against linear and rotational motion of the brain relative to the skull, thereby reducing the maximum velocity of the brain relative to the skull during an event and reducing the magnitude of acceleration experienced by the brain when the brain impacts the inside of the skull. Since acceleration has been identified as a significant factor in brain injury, said increase in CSF viscosity will prevent, or reduce the severity of, TBI.

It is recognized that said mechanics of the invention are presented as a simplified version of a complex system in order to demonstrate the concept of the invention. It is not the intent of said presentation to account for all potential impact geometries or any non-linear behavior associated with variables such as the soft substance of brain matter and surrounding tissue, the combined viscoelastic behavior of CSF with other soft tissue between the brain and the skull, the irregular surface contour of the outside of the brain, the cushion effect of CSF within spaces inside the brain, potential non-Newtonian fluid behaviors or Reynolds numbers of CSF, or the specific mechanics of any particular impact event.

It is also recognized that impact events may consist of both rotational acceleration and linear acceleration components. In said cases, the mechanical concepts depicted in both FIGS. 2 and 3 would be applicable.

Thermal regulation of CSF is one embodiment of the invention. The vast majority of CSF is composed of water and the effect of temperature on water viscosity is known (Viscopedia). At a normal human body temperature of 98.6° F. (37° C.) the viscosity of water is approximately 0.6913 mPa*s, and at a body temperature of 104° F. (40° C.) in a potential patient, the viscosity of water is approximately 0.6527 mPa*s. Since the vast majority of CSF is comprised of water, viscosity effects on water will correlate to similar effects on CSF. Therefore, thermal regulation of CSF in an active human body prior to an impact event offers the potential for an approximate increase of 6% in CSF viscosity to prevent or reduce the severity of TBI.

The stated claims include a reference to a “medically effective apparatus” to reduce the temperature of the head. Said claims are based upon a decrease in the temperature of the head and resulting increase in CSF viscosity, however, it does not depend on the particular apparatus used to achieve the temperature decrease. Specific embodiments of the invention are independent of the means of temperature regulation, and are not limited to any particular use or combination of conductive, convective, or radiative heat transfer. The invention utilizes proactive regulation of CSF temperature prior to an event in order to increase the viscosity of CSF and consequently prevent, or reduce the severity of, TBI. The protective benefit of the invention is independent of any mechanical benefit of the physical apparatus itself, because the invention utilizes the thermal regulation feature of the apparatus to modify an internal bodily characteristic in order to prevent, or reduce the severity of, TBI.

Temperature decreases as a result of the invention include those cases where, under conditions that would otherwise result in a higher-than-normal temperature of the head: (a) the invention maintains a constant temperature of the head, or (2) the invention minimizes the increase in the temperature of the head that would normally occur under such conditions.

One embodiment of the invention represents an improvement helmet technologies such as U.S. Pat. No. 7,827,620.

The stated claims include references to a “medically effective treatment”. The stated claims are not intended to specify the exact composition of any chemical components of such a treatment. The stated claims also include dependent claims related to certain specific embodiments of said treatment to achieve the intended effect on CSF viscosity.

The invention is not limited to any particular delivery method or combination of delivery methods of said treatment, even if certain methods of a particular embodiment may offer significant benefits over others in certain situations. One embodiment using intranasal delivery of a medically effective treatment may offer a more rapid adjustment of CSF composition to affect CSF viscosity due to the proximity of the application site to the brain. However, embodiments of the invention are not dependent on any particular delivery method since different delivery methods may be more appropriate than others based on the particular nature and duration of its effects, the particular risk scenarios of a specific patient, or other factors.

The daily CSF turnover rate of over 3 times allows for temporary changes to CSF to focus the treatment at those times when a patient is at a high risk of TBI. One embodiment of the invention provides temporary adjustments to CSF viscosity to be administered prior to periods of high risk, such as an athletic game, a motorcycle ride, or a combat mission. Another embodiment is administered during periods of high risk to continue the protective benefit until such time that the patient is no longer in a high risk scenario.

One data point supporting the ability to manipulate the viscosity of CSF is the wide normal range of CSF viscosity. Published normal values for CSF viscosity vary from 0.7 to 1.0 mPa*s at 37° C. Based on the resulting reduction in acceleration during impact events, the high end of the published normal viscosity range offers significant benefits over the middle of the range in preventing or reducing the severity of TBI. Specifically, the high end of the published normal range represents an 18% increase in CSF viscosity over the midpoint of the published normal range, and a 43% increase in CSF viscosity over the low end of the published normal range.

Research involving CSF viscosity as a diagnostic tool for meningitis confirmed a correlation between CSF protein concentrations and CSF viscosity, where a 16.6% increase in average viscosity was identified in the meningitis patients that had a 654% higher CSF protein concentration (Yetkin et al). Said correlation was confirmed in distinguishing the bacterial meningitis group from the aseptic meningitis group, which showed an 11.5% increase in CSF viscosity for the bacterial meningitis group, which had a 347% higher protein concentration. Another study (Brydon et al) confirmed increased CSF viscosity due to increased protein levels, although the increases identified in that study were dismissed as being insignificant because it did not resolve the objective of the study regarding failures of CSF flow through a shunt implant, even though it would affect the behavior of CSF during impact events.

Some specific embodiments of the invention are based on the nature of the human body to deploy defense mechanisms against injury. Several of the specific embodiments build on a correlation between repeated head impacts and the concentration of certain constituents of CSF. Studies of head trauma subjects have shown increased CSF levels of neurofilament light polypeptide, S-100B protein, glial fibrillary acidic protein, and tau protein (Zetterberg et al). However, said constituents are presented in the literature as a biomarker for use in diagnosis after the impact event, rather than recognition of a potential defense mechanism that could be deployed as a preventive measure. One embodiment that increases the concentration of tau protein in CSF may be deployed in combination with other known treatments intended to prevent the phosphorylation of tau to avoid potential side effects related to hyperphosphorylated tau protein, U.S. Pat. No. 9,415,063. Another embodiment incorporates aerobic exercise as part of the treatment in order to maximize CSF flow during and after the treatment to reduce stagnation-related side effects.

Other embodiments of the invention are based on the presence of existing constituents of normal CSF which are believed to be brain-derived, due to their higher normal concentrations in CSF versus in the bloodstream. Said embodiments seek to enhance the natural defensive properties of CSF by increasing the concentration of brain-derived constituents which include one or a combination of β-trace protein and cystatin C protein (Reiber).

Most CSF is produced by the choroid plexus, which serves as a barrier between the bloodstream and CSF, often referred to as the blood-brain barrier. One embodiment of the invention involves the manipulation of the behavior of the choroid plexus, which will allow the passage of agents from the bloodstream into CSF to result in an increased CSF viscosity. Another embodiment manipulates the behavior of the arachnoid villi, which are the primary path of CSF back into the bloodstream. Manipulation of the behavior of the choroid plexus or arachnoid villi offers the opportunity to manipulate the concentration of certain substances in the CSF, which offers the opportunity to increase the viscosity of CSF and thereby prevent or reduce the severity of TBI.

### REFERENCES

1. Brain Injury Association of America, www.biausa.org/bia-media-center.htm.
2. Meaney et. al, “Biomechanics of Concussion”, Clinical Sports Medicine, 2011 January; 30(1): 19-vii, PMCID: PMC3979340, NIHMSID: NIHMS304494.
3. The Mayo Clinic, http://www.mayoclinic.org/diseases-conditions/traumatic-brain-injury/basi-%20cs/definition/con-20029302?p=1
4. Guyton & Hall, “Textbook of Medical Physiology” (9^{th} ed.), Philadelphia: W.B. Saunders. ISBN 0-7216-5944-6.
5. Water viscosity table, Viscopedia, http://www.viscopedia.com/viscosity-tables/substances/water/.
6. Baeck et al., “The use of different CSF representations in a numerical head model and their effect on the results of FE head impact analyses”, 8^{th} European LS-DYNA Users Conference, Strasbourg, May 2011.
7. Yang et al., “Development of a Finite Element Head Model for the Study of Impact Head Injury”, BioMed Research International, Volume 2014, Article ID: 408278.
8. Eckner et al., “No Evidence for Cumulative Impact Effect on Concussion Injury Threshold”, Journal of Neurotrauma, 28:2079-2090 (October 2011), PMCID: PMC4346375.
9. Yetkin et al., “Cerebrospinal Fluid Viscosity: A Novel Diagnostic Measure for Acute Meningitis”, Southern Medical Journal, 2010; v103; 892-895.
10. Brydon et al., “Physical properties of CSF of relevance to shunt function. 1: The effect of protein upon CSF viscosity”, British Journal of Neurosurgery, 1995; 9; 639-644.
11. Zetterberg et al., “Biomarkers of mild traumatic brain injury in cerebrospinal fluid and blood”, Nature Reviews Neurology, April 2013; 9; 201-210, PMCID: PMC4513656.
12. H. Reiber, “Proteins in cerebrospinal fluid and blood: Barriers, flow rate, and source-related dynamics”, Restorative Neurology and Neuroscience 21 (2003) 79-96.

1. A method to prevent, or reduce the severity of, traumatic brain injury (TBI), said method comprising: (a) the preparation of a medically effective apparatus, and (b) administration of the apparatus to achieve an increase in the viscosity of cerebrospinal fluid (CSF).

2. The method of claim 1, where the medically effective apparatus is a helmet apparatus that reduces the temperature of the head.

3. A method to prevent, or reduce the severity of, traumatic brain injury (TBI), said method comprising: (a) the preparation of a medically effective treatment, and (b) administration of the treatment to achieve an increase in the viscosity of cerebrospinal fluid (CSF).

4. The method of claim 3, where a medically effective treatment results in an increase of concentration in CSF of any the following: acute inflammatory response proteins, neurofilament light polypeptide, S-100-B protein, glial fibrillary acidic protein, tau-protein, cystatin C protein, β-trace protein.

5. The method of claim 3, where a medically effective treatment manipulates the behavior of an existing bodily structure, which may include any of the following: choroid plexus, arachnoid villi.

6. The method of claim 3, where the medically effective treatment is administered prior to a patient's engagement in an activity with an elevated risk of head impacts.

7. The method of claim 3, where the medically effective treatment is administered during a patient's engagement in an activity with an elevated risk of head impacts.

8. The method of claim 3, where the CSF viscosity increase is in the range of 1% to 5%.

9. The method of claim 3, where the CSF viscosity increase is between 5% and 10%.

10. The method of claim 3, where the CSF viscosity increase is in the range of 10% to 20%.

11. The method of claim 3, where the CSF viscosity increase is between 20% and 30%.

12. The method of claim 3, where the CSF viscosity increase is in the range of 30% to 40%.

13. The method of claim 3, where the CSF viscosity increase is between 40% and 50%.

14. The method of claim 3, where the CSF viscosity increase is in the range of 50% to 60%.

15. The method of claim 3, where the CSF viscosity increase is between 60% and 70%.

16. The method of claim 3, where the CSF viscosity increase is in the range of 70% to 80%.

17. The method of claim 3, where the CSF viscosity increase is between 80% and 90%.

18. The method of claim 3, where the CSF viscosity increase is in the range of 90% to 100%.
